(19) United States
(12) Patent Application Publication
(10) Pub. No.: US 2008/0009079 A1
(43) Pub. Date: Jan. 10, 2008

(54) METHOD FOR FORMATION OF A STATIONARY PHASE IN AN IMMUNOADSORPTION WALL

(75) Inventor: Tsung-Hua Yang, Niaosong Township (TW)

Correspondence Address: WORKMAN NYDEGGER, 60 EAST SOUTH TEMPLE, 1000 EAGLE GATE TOWER, SALT LAKE CITY, UT, 84111, US

(73) Assignee: CHENG SHIU UNIVERSITY, Niaosong Township (TW)

(21) Appl. No.: 11/456,051

(22) Filed: Jul. 6, 2006

(51) Int. Cl. G01N 33/543

(52) U.S. Cl. 436/518

(57) ABSTRACT

A method for formation of a stationary phase in an immunoadsorption wall is provided. This method is a partially incomplete two-stage polymerization method and the resulting stationary phase consists of a supporting gel layer and a stacking gel layer. The method is mainly characterized by the change in the temperature and non-uniform concentration distribution of monomer acrylamide in the polymerization reaction system to produce the stationary phase superficially embedded with the immunoadsorbents. Three different ways are introduced to the formation of such a stationary phase including the pre-coupling process, the post-coupling process and the serial copolymerization process. The results of binding activity tests shows that the middle molecular toxin, beta-2-microglobulin, can be removed by the prepared stationary phases.

### BACKGROUND OF THE INVENTION

1. Field of Invention

The present invention relates to a method for formation of an auxiliary toxin-removal modality for hemodialysis, and more particularly, to a method for formation of a stationary phase in an immunoadsorption wall.

2. Related Art

Because of the regression or loss of renal functions, uremic patients have to rely on hemodialysis therapy to remove accumulated toxins to prolong lives. However, hemodialysis is not able to replace all of the renal physiological functions. Thus, long-term dialysis patients frequently develop various kinds of complications especially because hemodialysis fails to remove certain middle molecular weight toxins, e.g., β2-microglobulin (β-2M). Consequently, the quality of dialysis is seriously worsened, and the treatment lifespan is usually shortened.

The immunoadsorbent is a substance used to separate antigens from a mixture. The immunoadsorbent is prepared by coupling a slurry support matrix with certain antibodies; as the mixture flows through a column packed with the stationary immunoadsorbent, the antigens can be bound to the antibodies. Many researchers have attempted to prepare immunoadsorbents with high selectivity and affinity to remove β-2M (Mogi M, et al., 1993; Vallar L, et al., 1995). However, these approaches, because of the incompatibility between the immunoadsorbent and blood, seem unlikely to be put into long-term clinical practice (Vallar L, 1996).

In order to overcome the aforementioned problems of direct use of immunoadsorbent, an immunoadsorption wall has been suggested as a possible auxiliary toxin-removal modality in conjuction with hemodialysis filters. The concept of the immunoadsorption wall, which combines the principles of immunoisolation and immunoadsorption, is proposed to remove certain toxins accumulated in a patient's blood (Yang T H, et al., 1999). The immunoadsorption wall includes two parts: the first part is an immunoisolation barrier for improvement of the biocompatibility between blood and the toxin-removal modality; and the second part is the stationary phase that is capable of removing toxins by immunoadsorption. In theory, the immunoadsorption wall can be incorporated into a hemodialysis circuit, and the toxins can be removed from blood as cross-flow over the surface of the stationary phase is performed. A crude experimental model based on the tightly packing of immunoadsorbent has been employed to investigate its feasibility; however, the development of an immunoadsorption wall is apparently greatly impeded by the inefficient utilization of immunoadsorbent for formation of the stationary phase.

Polyacrylamide gel is a commonly used matrix in electrophoresis and is formed by the polymerization of monomers of acrylamide with monomers of a suitable bifunctional cross-linking agent. Polyacrylamide gel has the properties of chemical inertness, minimal diffusion effect, so that it may be considered as the medium of choice for serving as a matrix for a stationary phase in an immunoadsorption wall.

In light of above-mentioned, there is a need for improving the existing method for formation of the stationary phase in an immunoadsorption wall to advance the use of immunoadsorbent and optimize the formation of the stationary phase in an immunoadsorption wall. It is hoped that this stationary phase could be used to resolve some difficult problems arising from the accumulation of certain toxins not removed by other blood purification procedures.

### SUMMARY OF THE INVENTION

The present invention provides a method for formation of a stationary phase in an immunoadsorption wall which is capable of removing certain toxins from blood. In the present invention, a partially incomplete two-stage polymerization method is introduced for formation of a new stationary phase superficially embedded with immunoadsorbents.

The first embodiment of the present invention includes the steps of forming a supporting gel layer, loading a stacking solution with a plurality of immunoadsorbents onto top of the supporting gel layer, completing a sedimentation process of the immunoadsorbents while the stacking solution remains in an unpolymerization state, creating a non-uniform concentration of monomer within the settled immunoadsorbents, converting the remaining stacking solution to a polymerization state, and flushing away the unpolymerized portion to form a stacking gel layer with exposed immunoadsorbents. Furthermore, the step of creating a non-uniform concentration of monomer within the settled immunoadsorbents further includes a step of removing part of the supernatant and rinsing the surface of the remaining stacking solution with a buffer for dilution. The immunoadsorbents can be the complexes of support matrices and antibodies.

In the second embodiment of the present invention, a post-coupling process is used to replace the pre-coupling process for preparation of conventional slurry immunoadsorbents. This method includes the steps of forming a supporting gel layer, loading a stacking solution with a plurality of support matrices onto top of the supporting gel layer, completing a sedimentation process of the support matrices while the stacking solution remains in an unpolymerization state, creating a non-uniform concentration of monomer within the settled support matrices, converting the remaining stacking solution to a polymerization state to form a stacking gel layer with the exposed support matrices, and coupling a plurality of antibodies to the exposed support matrices. Furthermore, the step of creating a non-uniform concentration of monomer within the settled support matrices further includes a step of removing part of the supernatant and rinsing the surface of the remaining stacking solution with a buffer for dilution.

In the third embodiment of the present invention, the flushed-away immunoadsorbents are further recovered. This method includes the steps of forming a supporting gel layer, loading a stacking solution with a plurality of immunoadsorbents onto top of the supporting gel layer, completing a sedimentation process of the immunoadsorbents while the stacking solution remains in an unpolymerization state, creating a non-uniform concentration of monomer within the settled immunoadsorbents, and converting the remaining stacking solution to a polymerization state, flushing away the unpolymerized portion to form a stacking gel layer with exposed immunoadsorbents, and recovering the flushed-away immunoadsorbents. Furthermore, the step of creating a non-uniform concentration of monomer within the settled immunoadsorbents further includes a step of removing part of the supernatant and rinsing the surface of the remaining stacking solution with a buffer for dilution. The step of recovering the flushed-away immunoadsorbents further includes using the recovered immunoadsorbents to form another immunoadsorption wall.

The matrices of the supporting gel layer and the stacking gel layer both are polyacrylamide. The acrylamide concentration of the supporting gel layer is higher than the acrylamide concentration of the stacking gel layer to establish a molecular sieving effect between these two layers. The unpolymerization state may represent that polymerization is inhibited due to factors such as low temperature, addition of inhibitors, etc. The polymerization state may represent that polymerization is initiated due to factors such as high temperature, addition of initiators or catalysts, etc.

The stationary phase which includes the supporting gel layer and the stacking gel layer provided by the present invention is based on the polyacrylamide formed by the above-mentioned partially incomplete two-stage polymerization method. This method is mainly characterized by the change in the temperature and non-uniform concentration distribution of acrylamide in the polymerization reaction system to produce the stationary phase superficially embedded with the immunoadsorbents.

Further scope of the applicability of the invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given herein below for illustration only, and which thus is not limitative of the present invention, and wherein:

FIGS. 1A to 1E are flow diagrams according to a first embodiment of the present invention.

FIGS. 2A to 2F are flow diagrams according to a second embodiment of the present invention.

FIG. 3 is a diagram according to the result of binding activity test of the immunoadsorption wall provided by the first embodiment of the present invention.

FIG. 4 is a diagram according to the result of binding activity test of the immunoadsorption wall provided by the second embodiment of the present invention.

FIG. 5 is a diagram according to the result of binding activity test of the immunoadsorption wall provided by the third embodiment of the present invention.

### DETAILED DESCRIPTION OF THE INVENTION

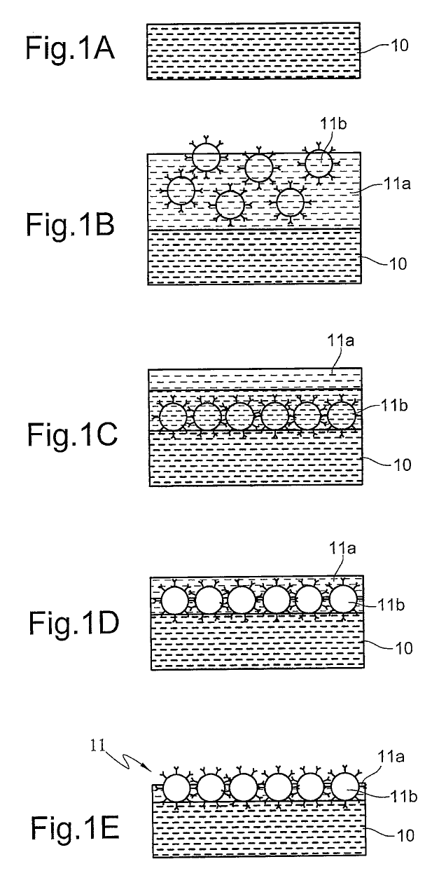

Please refer to FIGS. 1A to 1E of flowcharts of a first embodiment of the present invention. The first embodiment of the present invention consists of the following steps. First, form a supporting gel layer 10, as shown in FIG. 1A. Second, load a stacking solution 11a with a plurality of immunoadsorbents 11b onto top of the supporting gel layer 10, as shown in FIG. 1B. Third, complete the sedimentation process of the immunoadsorbents 11b while the stacking solution 11a remains in an unpolymerization state, as shown in FIG. 1C. Fourth, create a non-uniform concentration distribution of monomer within the settled immunoadsorbents as shown in FIG. 1D. Fifth, convert the remaining stacking solution 11a to a polymerization state. Finally, flush away the unpolymerized portion to form a stacking gel layer 11 with the exposed immunoadsorbents 11b as shown in FIG. 1E. Furthermore, the step of creating a non-uniform concentration of monomer within the settled immunoadsorbents further includes a step of removing part of the supernatant of the stacking solution 11a and rinsing the surface of the remained stacking solution 11a with a buffer for dilution. The supporting gel layer 10 and the stacking gel layer 11 both are polyacrylamide. The acrylamide concentration of the supporting gel layer 10 is higher than the acrylamide concentration of the stacking gel layer 11. The unpolymerization state is the stacking solution 11a at inhibitive polymerization temperature. The polymerization state is the stacking solution 11a at initiative polymerization temperature.

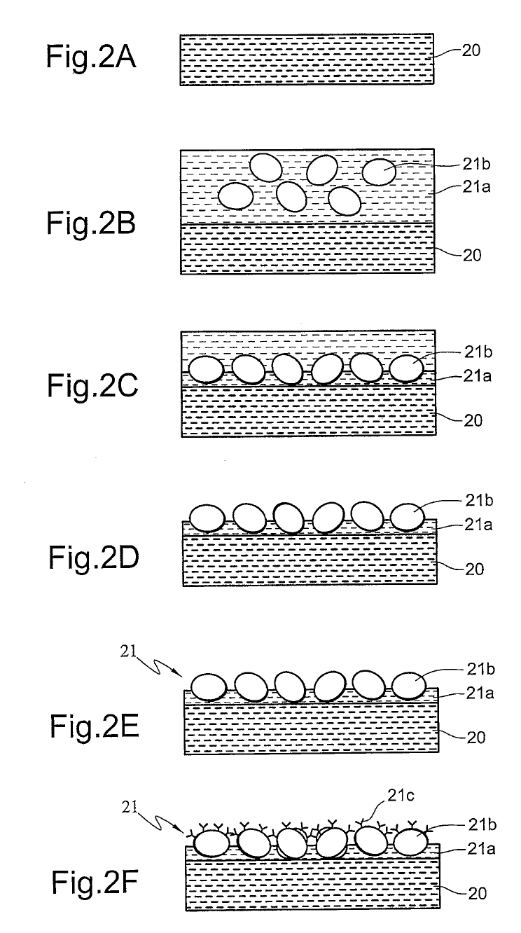

Please refer to FIGS. 2A to 2F of flowcharts of a second embodiment of the present invention. A post-coupling process is used to replace the pre-coupling process for preparation of conventional slurry immunoadsorbents. The second embodiment of the present invention consists of the following steps. First, form a supporting gel layer 20 as shown in FIG. 2A. Second, load a stacking solution 21a with a plurality of support matrices 21b onto top of the supporting gel layer 20 as shown in FIG. 2B. Third, complete a sedimentation process of the support matrices 21b while the stacking solution 21a remains in an unpolymerization state as shown in FIG. 2C. Fourth, create a non-uniform concentration distribution of monomer within the support matrices 21b as shown in FIG. 2D. Fifth, convert the remaining stacking solution 21a to a polymerization state and flush away the unpolymerized portion so as to form a stacking gel layer 21 with the exposed support matrices 21b as shown in FIG. 2E. Finally, couple a plurality of antibodies 21c to the exposed support matrices 21b. Furthermore, the step of creating a non-uniform concentration of monomer within the settled support matrices further includes a step of removing part of the supernatant of the stacking solution 21a and rinsing the surface of the remained stacking solution 21a with a buffer for dilution.

In a third embodiment of the present invention, the flushed-away immunoadsorbents after formation of the stacking gel layer are further recovered. And then the recovered immunoadsorbents can be used to form another immunoadsorption wall.

The invention will now be described with reference to the following specific Examples which are illustrative, and not limiting, of the invention.

### EXAMPLE 1

### The Pre-Coupling Process for Formation of a Stationary Phase by the Partially Incomplete Two-Stage Polymerization Method

### Materials

Acrylamide-bisacrylamide (29:1) solution (Sigma, St. Louis, Mo., U.S.A.) is prepared by dissolving 29 g of acrylamide and 1.0 g of bisacrylamide in a total volume of 100 mL of water. Ammonium persulfate (10%, w/v) (Sigma) serving as the initiator of polymerization is made fresh just before use. N,N,N′,N′-tetramethylethylenediamine (TEMED) (Bio-Rad, Hercules, Calif., U.S.A.) is added as accelerator of the polymerization process without pretreatment. The immunoadsorbents is prepared by coupling Rabbit CNBr-activated Sepharose 4B (Amersham Biosciences, Piscataway, N.J., U.S.A.) with anti-β2-microglobulin (β-2M) antibodies (Dako, Glostrup, Denmark). The immunoadsorbent thus prepared is stored at 4° C. in 10 mM sodium phosphate buffer with 0.15 M NaCl (pH 7.4) (PBS) (Sigma) containing 0.02% NaN₃ (Merck, Whitehouse Station, N.J., U.S.A.). Human β-2M solution is fractionated from urine of hemodialysis patients as reported (Vallar L, et al., 1995). The determination of β-2 M level is made by enzyme immunoassay kit (BioCheck, Foster City, Calif., U.S.A.). Regeneration buffer is 0.3 M (pH 2.8) glycine-HCl (Sigma).

### Methods

In the first embodiment, a stationary phase comprising a supporting gel layer (total concentration of monomer, T %=15%) and a stacking gel layer (T %=7.5%) with immobilized immunoadsorbents is formed in a thick-wall 25-mm diameter glass tube. The present invention is a two-stage polymerization method. The first stage is polymerization of higher content acrylamide to form a more restrictive supporting gel layer. The supporting gel layer is formed by polymerization of 5 mL acrylamide-bisacrylamide (29:1) polymer solution (acrylamide-bisacrylamide=15%) containing a catalyst system of 20 μL ammonium persulfate (w/v=10%) and 5 μL N,N,N′,N′-tetramethylenediamine (TEMED). At the beginning of the second stage, the temperature of the reaction system is lowered and maintained at 0° C. by an ice-water bath. Then, lower acrylamide content polymer solution (the stacking solution) containing 2 mL of immunoadsorbents is added on top of the supporting gel layer. The stacking solution includes 5 mL acrylamide-bisacrylamide (29:1) polymer solution (acrylamide-bisacrylamide=7.5%), 20 μL ammonium persulfate (w/v=10%) and 5 μL N,N,N′,N′-tetramethylenediamine (TEMED). The immunoadsorbents of the first embodiment are the complexes made by coupling CNBr-activated Sepharose 4B with rabbit anti-β-2M antibodies. Because the temperature of the reaction system is maintained at 0° C., the polymerization of acrylamide-bisacrylamide polymer solution could be inhibited. Therefore, the stacking solution remains in an unpolymerization state and the sedimentation of the immunoadsorbents could be completed. Then, part of the supernatant of the stacking solution is removed and the surface of the stacking solution is rinsed with PBS buffer for dilution. Subsequently, the reaction system returned to room temperature (c. 30° C.) and the polymerization reaction of the stacking solution initiated spontaneously. Accordingly, antihuman β-2M immunoadsorbents are copolymerized with acrylamide to form a stacking gel layer. Actually, the upper portion of the remaining stacking solution did not polymerize due to previous dilution and the unpolymerized portion is washed away by a flush of regeneration buffer once the inner portion is thoroughly polymerized. As a result, a stationary phase superficially embedded with the immunoadsorbents is formed. To prevent dehydration and potential microbial contamination, the surface of the stationary phase is supplied with PBS containing 0.02% NaN₃.

### Binding Activity Test

The prepared stationary phase is then tested for its adsorption capacity by adding 3 mL of β-2M in pH 7.4 phosphate buffer with concentration level of approximately 180 μg/mL at 37° C. Samples (20 μL) are collected from the supernatant at 0, 1, 2, 3, 4, and 5 h, respectively (n=3). Further, the binding activity of the stationary phase is regenerated by washing three times with glycine-HCl solution. Three adsorption-desorption cycles are made. All immunoadsorption cycles and quantitative assays are carried out in the same operating conditions.

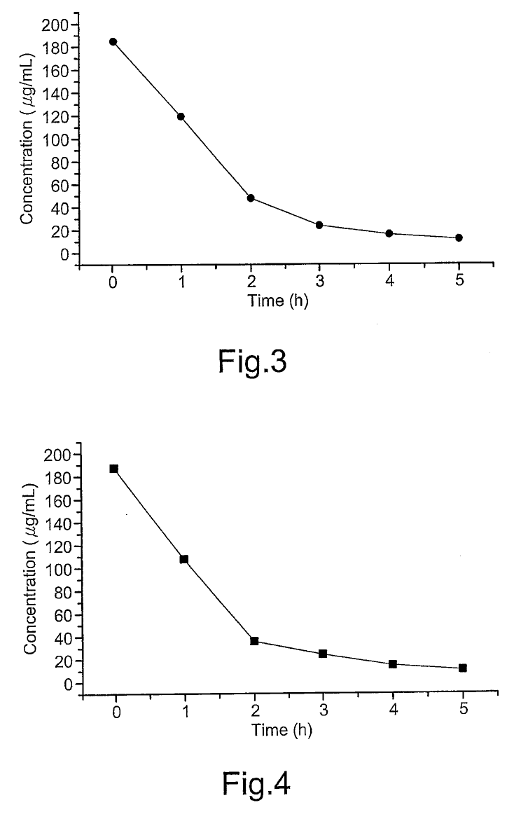

Referring to FIG. 3, the result of binding activity test of the immunoadsorption wall provided by the first embodiment is shown. The level of β-2M decreases rapidly with time until saturation is achieved after about 3 hours and then gradually decreases to ca. 10 μg/mL. Thus, it shows that the activities of the immunoadsorbents are maintained and demonstrates the superior capability of this stationary phase to remove β-2M.

### EXAMPLE 2

### The Post-Coupling Process for the Formation of a Stationary Phase by the Partially Incomplete Two-Stage Polymerization Method

In the second embodiment, the formation of a stationary phase is still the two-stage polymerization method. But Rabbit anti-β2-microglobulin (β-2M) antibodies are not coupled with CNBr-activated Sepharose 4B at the start. Rather, CNBr-activated Sepharose 4B are alone embedded in the finished stationary phase, and then β-2M antibodies are coupled with CNBr-activated Sepharose 4B settled on the surface of the finished stationary phase.

### Methods

In the second embodiment, the supporting gel layer is formed by polymerization of 5 mL acrylamide-bisacrylamide (29:1) polymer solution (acrylamide-bisacrylamide=15%) containing a catalyst system of 20 μL ammonium persulfate (w/v=10%) and 5 μL N,N,N′,N′-tetramethylenediamine (TEMED). Subsequently, the temperature of the reaction system is lowered and maintained at 0° C. by an ice-water bath. Then, the stacking solution containing 0.3 g CNBr-activated Sepharose 4B is added on top of the supporting gel layer. The stacking solution included 5 mL acrylamide-bisacrylamide (29:1) polymer solution (acrylamide-bisacrylamide=7.5%), 20 μL ammonium persulfate (w/v=10%) and 5 μL N,N,N′,N′-tetramethylenediamine (TEMED). The stacking solution remains in an unpolymerization state (0° C.), and the sedimentation of CNBr-activated Sepharose 4B are completed. Then, part of the supernatant of the stacking solution is removed and the surface of the stacking solution is rinsed with PBS buffer for dilution. Subsequently, the reaction system returned to room temperature, which brought the stacking solution to the polymerization state. As a result, a stationary phase superficially embedded with CNBr-activated Sepharose 4B is formed. Then P-2M antibodies are loaded onto the surface of the stationary phase and coupled with CNBr-activated Sepharose 4B. Then, 0.1M Tris-HCl solution (pH 8.0) is added to block the coupling reaction. Finally, the surface of the stationary phase is alternately washed with 0.1M acetate buffer (pH 4.0) and 0.1M Tris-HCl solution (pH 8.0) for at least three cycles. The post-coupling process of the formation of a stationary phase is completed. In addition, the activation of Sepharose 4B can be done after finishing the stationary phase embedded with Sepharose 4B.

### Binding Activity Test

The prepared stationary phase is then tested for its adsorption capacity by adding 3 mL of P-2M in pH 7.4 phosphate buffer with concentration level of approximately 180 μg/mL at 37° C. Samples (20 μL) are collected from the supernatant at 0, 1, 2, 3, 4, and 5 h, respectively (n=3). Further, the binding activity of the stationary phase is regenerated by washing three times with glycine-HCl solution. Three adsorption-desorption cycles are made. All immunoadsorption cycles and quantitative assays are carried out in the same operating conditions.

Referring to FIG. 4, the result of binding activity test of the immunoadsorption wall provided by the second embodiment has been shown. Similarly, the level of β-2M (concentration) decreases rapidly with time until saturation is achieved after about 3 hours and then gradually decreases to ca. 10 μg/mL. Thus, it shows that the activities of the antibodies are maintained and demonstrates the superior capability of this stationary phase to remove β-2M.

### EXAMPLE 3

### A Serial Copolymerization Method for Manufacturing Stationary Phases in Immunoadsorption Walls

In the third embodiment, in order to enhance the utilization efficiency of immunoadsorbents, the flushed-away immunoadsorbents are further recovered, and the copolymerization is conducted in series to produce three consecutive immunoadsorption walls.

### Methods

In the third embodiment, the supporting gel layer is formed by polymerization of 5 mL acrylamide-bisacrylamide (29:1) polymer solution (acrylamide-bisacrylamide=15%) containing a catalyst system of 20 μL ammonium persulfate (w/v=10%) and 5 μL N,N,N′,N′-tetramethylenediamine (TEMED). Then, the temperature of the reaction system is lowered and maintained at 0° C. by an ice-water bath and lower acrylamide content polymer solution (the stacking solution) containing 2 mL of immunoadsorbents is added on top of the supporting gel layer. The stacking solution includes 5 mL acrylamide-bisacrylamide (29:1) polymer solution (acrylamide-bisacrylamide=7.5%), 20 μL ammonium persulfate (w/v=10%) and 5 μL N,N,N′,N′-tetramethylenediamine (TEMED). Further, remove the supernatant of the stacking solution and rinse the surface of the remained stacking solution with buffer for dilution, and let the temperature of the reaction system return to room temperature (e.g. 30° C.) so that the polymerization reaction initiates spontaneously to form a more porous stacking gel layer. Once the inner portion is thoroughly polymerized, flush away the unpolymerized portion of the stacking gel with neutral phosphate buffer (The above-mentioned steps can refer to the first embodiment). The flushed-away immunoadsorbents are extensively washed with three cycles of high and low pH buffers, and then kept at 4° C. for subsequent recycled use. Finally, make up the required amount of immunoadsorbents with an intact one and repeat the above steps to manufacture additional immunoadsorption walls. In the third embodiment, three consecutive immunoadsorption walls using antihuman β-2M immunoadsorbents are made and labeled Nos. 1-3 in serial order. The immunoadsorption walls thus prepared is supplied with PBS buffer for prevention of dehydration and kept at 4° C.

In addition, the uncoupling antibodies in the second embodiment also can be recovered and applied to form another immunoadsorption wall.

### Binding Activity Test

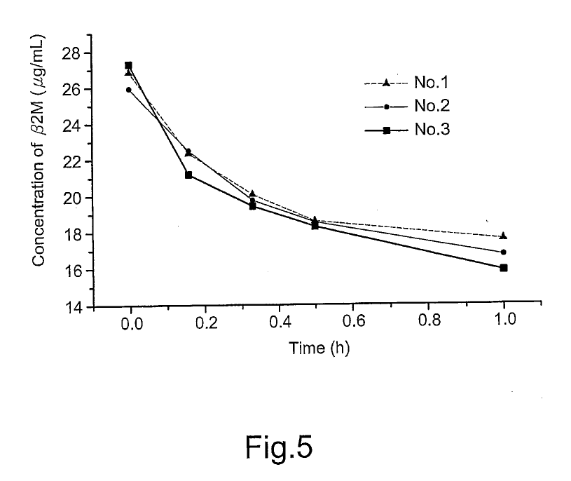

The prepared immunoadsorption walls are then tested for their immunoadsorption efficacy by adding 1 mL of P-2M in pH 7.4 phosphate buffer with concentration levels of approximately 30 μg/mL at 37° C. Samples (20 μL) are collected from the supernatant at 0, 0.16, 0.33, 0.5, 1 h, respectively. The time course change of the β-2M levels is assayed by the enzyme-linked immunoassay. Referring to FIG. 5, it shows the time course change of β-2M levels (concentrations) in the immunoadsorption tests. In general, the removal patterns are similar for the three immunoadsorption walls (nos. 1-3), and the concentration of β-2M decreases from 26.66 to 16.68 μg/mL on the average.

### CONCLUSIONS

The method for formation of a stationary phase in an immunoadsorption wall of the present invention is mainly a two-stage polymerization reaction. Further, in order to avoid shielding immunoadsorbents from toxins by polyacrylamide, the most important key step is the intentional dilution of the surface of the stacking solution settled with the immunoadsorbents in the second stage. When the surface of the stacking solution settled with the immunoadsorbents is rinsed with buffer, a non-uniform concentration distribution of acrylamide would be created. Thus, copolymerization of acrylamide with immunoadsorbent in the upper portion will not begin under relatively low concentration of monomers while the copolymerization in the inner portion proceeds. Relying on the stationary phase provided by the present invention, the toxin can immediately contact with the surface of the stationary phase and is bound to the exposed ligands of the immunoadsorbents with little or no hindrance until saturation. Moreover, the discontinuous porous structure of the stationary phase could produce a considerable molecular sieving effect, thereby preventing leakage of some essential components. On the other hand, the procedure for coupling CNBr-activated support matrices with antibody could be done after finishing the stationary phase embedded with the CNBr-activated carriers (referring to the second embodiment). Moreover, the washed-away immunoadsorbents or antibodies during preparation could be pooled for recycling (referring to the third embodiment).

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected.

What is claimed is:

1. A method for formation of a stationary phase in an immunoadsorption wall, comprising the steps of:
forming a supporting gel layer;
loading a stacking solution with a plurality of immunoadsorbents onto top of the supporting gel layer;
completing a sedimentation process of the immunoadsorbents while the stacking solution remains in an unpolymerization state;
creating a non-uniform concentration distribution of monomer within the settled immunoadsorbents;
converting the remaining stacking solution to a polymerization state; and
flushing away the unpolymerized portion to form a stacking gel layer with exposed immunoadsorbents.

2. The method as claimed in claim 1, wherein the step of creating a non-uniform concentration distribution of monomer within the settled immunoadsorbents further comprises a step of:
removing part of the supernatant and rinsing the surface of the remaining stacking solution with a buffer for dilution.

3. The method as claimed in claim 1, wherein the immunoadsorbents are the complexes of support matrices and antibodies.

4. The method as claimed in claim 3, wherein the support matrices are CNBr-activated Sepharose 4B.

5. The method as claimed in claim 3, wherein the antibodies are anti-β2-microglobulin antibodies.

6. The method as claimed in claim 1, wherein the supporting gel layer and the stacking gel layer both are polyacrylamide gel, the acrylamide concentration of the supporting gel layer is higher than the acrylamide concentration of the stacking gel layer.

7. The method as claimed in claim 1, wherein the unpolymerization state is the stacking solution at an inhibitive polymerization temperature.

8. The method as claimed in claim 1, wherein the polymerization state is the stacking solution at an initiative polymerization temperature.

9. The method as claimed in claim 1, further comprising a step of:
recovering the immunoadsorbents after formation of a stacking gel layer.

10. The method as claimed in claim 9, further comprising a step of:
using the recovered immunoadsorbents to form another immunoadsorption wall.

11. A method for formation of a stationary phase in an immunoadsorption wall, comprising the steps of:
forming a supporting gel layer;
loading a stacking solution with a plurality of support matrices onto top of the supporting gel layer;
completing a sedimentation process of the support matrices while the stacking solution is remained an unpolymerization state;
creating a non-uniform concentration distribution of monomer within the settled support matrices;
converting the remaining stacking solution to a polymerization state to form a stacking gel layer with the exposed support matrices, and coupling a plurality of antibodies to the exposed support matrices.

12. The method as claimed in claim 11, wherein the step of creating a non-uniform concentration distribution of monomer within the settled support matrices further comprising a step of:
removing part of the supernatant and rinsing the surface of the remaining stacking solution with a buffer for dilution.

13. The method as claimed in claim 11, wherein the support matrices are CNBr-activated Sepharose 4B.

14. The method as claimed in claim 11, wherein the antibodies are anti-β2-microglobulin antibodies.

15. The method as claimed in claim 11, wherein the supporting gel layer and the stacking gel layer both are polyacrylamide gel, the acrylamide concentration of the supporting gel layer is higher than the acrylamide concentration of the stacking gel layer.

16. The method as claimed in claim 11, wherein the unpolymerization state is the stacking solution at an inhibitive polymerization temperature.

17. The method as claimed in claim 11, wherein the polymerization state is the stacking solution at an initiative polymerization temperature.

18. The method as claimed in claim 11, further comprising a step of:
recovering the uncoupling antibodies.

19. The method as claimed in claim 18, further comprising a step of:
using the recovered antibodies to form another immunoadsorption wall.
